(19) United States
(12) Patent Application Publication
(10) Pub. No.: US 2014/0348795 A1
(43) Pub. Date: Nov. 27, 2014

(54) NUTRITIONAL BEVERAGE POWDER AND METHOD OF MAKING SAME

(71) Applicants: Helmut Wyzisk, Celebration, FL (US); Michael J. Sloggett, Plano, TX (US)

(72) Inventors: Helmut Wyzisk, Celebration, FL (US); Michael J. Sloggett, Plano, TX (US)

(21) Appl. No.: 14/288,051

(22) Filed: May 27, 2014

Related U.S. Application Data
(60) Provisional application No. 61/872,433, filed on Aug. 30, 2013.
(60) Provisional application No. 61/827,368, filed on May 24, 2013.

(51) Int. Cl. A61K 38/39; A61K 35/74; A61K 31/198; A61K 31/555; A61K 36/906; A61K 36/42; A61K 36/45; A61K 36/05; A61K 31/352; A61K 9/00; A61K 36/88

(52) U.S. Cl. CPC A61K 38/39; A61K 9/0095; A61K 35/744; A61K 31/198; A61K 31/555; A61K 36/88; A61K 36/42; A61K 36/45; A61K 35/748; A61K 36/05; A61K 31/352; A61K 36/906
USPC 424/93.3

(57) ABSTRACT

A nutraceutical compound designed to promote cellular health, cleanse and replenish a body's gastro-intestinal tract and system, balance a body's natural defense system, keep the immune system high, and keep intestinal bacteria feeding on the correct enzymes is disclosed. More specifically, a nutraceutical compound that includes glutathione, carnosin, B vitamins, amino acids, probiotics, aloe vera, chlorella, spirulina, fruit powders, superfoods, and minerals is disclosed.

### CROSS-REFERENCE TO RELATED APPLICATIONS

This application claims the benefit of U.S. Provisional Application Ser. No. 61/827,368, filed on May 24, 2013, titled NUTRITIONAL BEVERAGE POWDER AND METHOD OF MAKING SAME and of U.S. Provisional Application Ser. No. 61/872,433, filed on Aug. 30, 2013, titled NUTRITIONAL BEVERAGE POWDER AND METHOD OF MAKING SAME.

### FIELD OF THE DISCLOSURE

The present disclosure relates to a nutraceutical compound in the form of a soluble beverage powder, designed to target cellular biosynthesis.

### BACKGROUND OF THE INVENTION

Cellular deficiencies and their cumulative effects have very profound consequences on our health and well-being. Aging and disease have always been presented as different processes when in fact, they are synergistic; both starting as slow, insidious malnutrition of the cell. Failure to address cellular deficiencies leads to chronic illness and premature organ aging.

Many symptoms have become common in our society, such as chronic fatigue, anxiety and insomnia, and have been accepted as a normal part of everyday life. Radiant health and vitality seem to be out of reach and even avid seekers of health are unable to sift through the litany of medical journals and terminology. Issues such as graying and thinning hair, fatigue, arthritic joints, and brittle nails are accepted as inevitable signs of aging, but in reality, these symptoms are actually outward manifestations of multiple, internal nutritional deficiencies—specifically, deficiencies at the cellular level.

Additionally, individuals with diseases that causes wasting syndrome, such as cancer and HIV/AIDS, experience wasting because their bodies are shutting down and cannot absorb nutrients. This wasting results in accelerated aging for people who are already fighting severe disease.

### SUMMARY OF THE INVENTION

Disclosed is a formula for a nutraceutical compound in the form of a beverage powder that is designed to treat wasting syndrome and address subcritical levels of wasting by promoting cellular biosynthesis. The premise underlying the formula and resulting compound is to fulfill the requirements of the cellular machinery so that the body can accelerate the generation of new cells and regeneration of old cells. The formula is implemented using advanced microencapsulation of ingredients to create an optimum rejuvenation action on the cellular level. The compound created using this formula includes various precursors, vitamins, minerals, probiotics, prebiotics, and superfoods that can interact to establish the benefits of: glutathione, an anti-oxidant; Carnosine, a compound that support active metabolism; and collagen, a molecule that provides structural integrity and scaffolding for skin, bones, and joints. The disclosed formula includes a comprehensive profile of vitamins, minerals, anti-oxidants, and blood purifiers along with aloe vera to boost bioavailability. The formula is made of natural ingredients, flavors, and sweeteners, and can establish new levels of micronutrient balance to positively impact physical, psychological, and mental wellbeing in a user. The nutraceutical compound created by way of the formula provides nutritional support plus micronutrients and antioxidants, and is engineered to establish broad-scale nutritional balance at the individual cell level, across every cell in a user's body.

### DETAILED DESCRIPTION

The nutritional compound created by way of the formula disclosed herein may comprise, consist of, or consist essentially of the ingredients described herein, in the amounts described herein or in other amounts, as well as any additional or optional ingredients, components or limitations useful in a nutraceutical application. All percentages, parts and ratios used in this disclosure are by weight of the total compound, unless otherwise specified. All numerical ranges as used herein are intended and understood to be approximate, unless otherwise specified. All references to singular characteristics or limitations of the formula shall include the corresponding plural characteristic or limitation (and vice versa), unless otherwise specified or clearly implied by the context. All methods and processes described herein and the steps of each can be performed in any order, unless otherwise specified or clearly implied by the context.

Disclosed is a formula for a nutraceutical compound in the form of a beverage powder that is designed to treat wasting syndrome and address subcritical levels of wasting by promoting cellular biosynthesis. The premise underlying the formula and resulting compound is to fulfill the requirements of the cellular machinery so that the body can accelerate the generation of new cells and regeneration of old cells. To improve cellular turnover, the disclosed formula provides cellular machinery with specific amino acids, vitamins, minerals, probiotics, prebiotics, and superfoods that are rarely found in an average person's diet. By adding the disclosed compounds, the body can better absorb nutrients, improve cellular health, rebuild tissue, and diminish levels of wasting.

The formula disclosed herein may contain a number of ingredients. In one embodiment, the formula includes glutathione precursors, such as cysteine, glycine, and glutamic acid, beta-alanine, collagen peptides, vitamins, minerals, probiotics, prebiotics, and superfoods, such as chlorella, spirulina, and aloe vera, among other things. The disclosed formula may further contain other optional ingredients or components, such as, but not limited to, alpha lipoic acid, that may serve as further nutraceutical components, or that may modify the physical, chemical or aesthetic characteristics of the compound. Many such optional ingredients that are known in the art may be used in the formula disclosed herein, provided that such optional ingredients are compatible with those essential ingredients described herein and are safe and suitable for oral human consumption.

Glutathione is an antioxidant that assists the liver in neutralizing harmful, toxic substances. It cleanses and detoxifies the liver, assists the mitochondria of each cell, maintains cellular energy and integrity, and slows down cellular aging. However, glutathione is not well absorbed across the gastrointestinal (GI) tract and, therefore, the most effective method of increasing glutathione in the body, is to provide the body with the proper glutathione precursors so that it can, in turn, synthesize those precursors into glutathione. The precursors to glutathione are the amino acids cysteine, glycine, and glutamic acid.

Beta alanine is a precursor to carnosine, a dipeptide of the amino acids beta alanine and histidine, a powerful bioactive compound that helps support active metabolism by increasing lean muscle mass, strength, and stamina and by increasing rapid muscle recovery. Carnosine is a dipeptide of the amino acids beta alanine and histidine. Carnosine also lowers elevated blood sugar levels. This leads to stabilizing blood glucose levels over time, helping to prevent diseases like diabetes.

Collagen peptides are precursors to collagen, a complex molecule synthesized by the body to provide structural integrity and scaffolding for youthful skin, strong bones, and flexible joints.

An integral part of the disclosed formula is an array of vitamins and minerals, which are critical for a body's enzymes and co-enzymes to efficiently catalyze and accelerate biosynthesis needed to rebuild and replace old cells and to build new cells. Vitamins B1, B3, B6 and B12 make up an important class of nutrients that directly affects a person's mood, well-being and cognition, including memory and attention span. Deficiencies of the B-complex are strongly linked to cognitive dysfunction including brain fog, poor memory, poor attention span, mood swings, insomnia, irritability and anxiety. Through micro-encapsulation of the oil soluble vitamins A, D, & E, those oil soluble vitamins have been transformed into a water-soluble solution to make applicable in a beverage powder compound. Vitamins A, D, & E, combined with N-Acetyl Carnitine, are used to dramatically increase the melanin production, thus helping users see improved hair, eyes, skin and nails.

Minerals are key ingredients in the production of several hormones including the metabolic regulator known as thyroid hormone. Foods today are chronically devoid of vital minerals such as zinc and selenium because they are grown in depleted soil. Minerals are critical cofactors involved in hundreds of chemical reactions within the body, including the regulation of blood sugar, production of hormones and neurotransmitters and production of energy. Without these co-factors, the body is unable to produce adequate amounts of thyroid hormone as well as many other key substances like the mood regulating neurotransmitter, serotonin. This leads to weight gain, low mood and blood sugar issues. The formula disclosed herein is a powerful catalyst for weight loss because it includes selenium and a trace mineral blend that naturally boosts thyroid hormone production, in turn boosting metabolism.

The GI tract is one of the most important systems in human bodies because many other bodily functions emanate and rely upon the GI tract to enable their proper function. The formula disclosed herein has a probiotic blend of the digestive aids Lactobacillus acidophilus and Bifidobacterium bifidum that combine to repopulate the small and large intestine microflora. This is an extremely potent dose and will help to create a healthy GI tract by balancing the microbiome in the gut. The disclosed formula also includes a probiotic blend of soluble fibers that provide targeted energy for the healthy gut microflora to prosper and restore GI balance.

The word “superfoods” refers to foods that are known to have very high levels of phytochemicals and very low levels of substances generally regarded as unhealthy, such as saturated fats, additives and contaminants. The superfoods content in the formula disclosed herein includes chlorella, spirulina, and aloe-vera powder, providing detoxifying and cleansing benefits.

Chlorella is an algae high in protein. Studies have shown it has the ability to draw toxins from the GI tract, and because of its ability to bind with certain metals, has become known as an effective heavy metal detoxifier. Toxins build up in the body from poor diet, stress and environmental factors, causing the blood to become acidic and throwing off its vital pH balance. This imbalance causes muscle soreness, fibromyalgia and common aches and pains. The acidity also wears away at the joints and sets the foundation for the development of ailments such as arthritis and diabetes. Spirulina is a potent blood cleanser and purifier that promote alkalinity, keeping the blood's acidic levels in check and creating balanced pH levels. Also present in the disclosed formula are a number of other fruit and vegetable extracts, further enhancing this detoxifying effect.

Micronutrients, including vitamins, minerals, antioxidants, amino acids and other compounds, work synergistically, and there exists an important and delicate balance of nutrients. The disclosed formula can facilitate a balance of micronutrients. Taking too much of one nutrient may induce a deficiency in another nutrient by disturbing the biochemical balance they have with each other. For example, a person may load up on zinc supplements to help prevent catching a cold, which can cause some harm in the form of a copper deficiency. Zinc and copper levels must be in harmonious balance in a human body. If there is too much zinc and not enough copper, neurological problems may arise. The disclosed formula facilitates targeted supplementation to correct nutritional deficiencies specific to a user.

The combination of Probiotics, Bromeleain, Spirulina, Watermelon fruit powder, Blueberry fruit powder, Chlorella Powder, and Aloe Vera helps to cleanse and replenish the body's GI tract and system, balance the body's natural defense system, keep the immune system high, and keep the correct intestinal bacteria feeding on the correct enzymes to aid in easier replication of those bacteria.

Alpha-lipoic acid is a versatile antioxidant that functions in both water and fat, helping protect against a wide range of issues from heart disease to cataracts, as well as cognitive dysfunction and the aging of the body in general. Alpha-lipoic acid, also known as thioctic acid, also affects gene expression. Human bodies all have genes that make us susceptible to certain diseases; however, this mere presence alone doesn't mean the gene will express itself. Genes need to be “turned on,” or activated, before they actually exert their effects. Alpha-lipoic acid can activate good genes and deactivate bad ones.

Regardless of the underlying cause, a functional nutrient deficiency will result in defective metabolic activity. The biochemical pathways that depend on a specific nutrient will be compromised when a functional deficiency of that nutrient exists. A deficiency may exist several months or years before clinical symptoms of overt deficiency are seen. For example, the clinical presentation of a gross vitamin C deficiency is scurvy, but before this extreme deficiency is seen, subclinical deficiency exists and the clinical presentation is much more elusive. For a “subclinical” vitamin C deficiency, for example, there may be symptoms of fatigue or reduced immunity. Treating subclinical deficiencies before they become overtly clinical in classic deficiency disease is ideal.

The formula disclosed herein may be delivered in the form of a powder that a user may mix with the liquid of their choice (for example, water). Benefits of using the compound created by way of the formula include, but are not limited to, restoration, revitalization, and rejuvenation of the user's body on a cellular level; support of cardiovascular health; encouragement of nutrient absorption; improvement of GI tract function; assistance with immune function; increased energy and vitality; boosted focus and mental clarity; promotion of skin and hair benefits; and enhancement of lean muscle mass, among other benefits. In some embodiments, the disclosed formula includes protein. Alternatively, in some embodiments, the formula may not include protein. Additionally, the beverage powder could be sold pre-mixed with a liquid as a ready-to-drink beverage.

The formula disclosed herein may include natural or artificial flavors, or both, in some embodiments. The particular type and amount of flavor used in the formula may vary, and may depend on the desired flavor impression and the form of the flavor component(s) used. The type and amount of flavor to be used will be apparent to one skilled in the art and will depend on the desired flavor intensity.

The disclosed formula may be comprised of a number of ingredients, as described in more detail in the examples below. The ingredients and amounts used are listed here as examples only, and the formula may vary in some embodiments.

In one example, the serving size of the compound created by way of the disclosed formula may be 22 g. The compound may include the following ingredients: Beta Alanine, Malic Acid, Bromeleain, N-acetyl L-Carnitine, Isoleucine, Leucine, Aloe Vera Powder, Spirulina, Valine, Probiotic Blend, Lunasin, Ginger Root Powder, Carrot Powder, Watermelon Fruit Powder, Blueberry Fruit Powder, Chlorella Powder, Kelp Powder, Pomegranate Fruit Powder, PABA, Quercetin, and Glutathione. Other ingredients may include Fructose, Maltodextrin, Citric Acid, Natural Flavors, Stevia Extract, Rebaudioside A (Stevia Leaf Extract), FD&C Yellow #6, FD&C Red #40. Table 1 below demonstrates an example amount per serving and percent daily value (based on a 2,000 calorie diet) of the formula.

In some embodiments, the formula may be sugar-free. For example, the fructose may be omitted from the formula. The sugar-free embodiments of the formula may be used by individuals who are diabetic or have other dietary restrictions that do not permit consumption of sugars or may be used by those who are simply trying to reduce sugar intake. Alternative sweeteners, for example, stevia extract, may be included in sugar-free embodiments. The formula may also include ingredients to add fiber.

Sugar-free embodiments may be varied flavors, as described herein. In some embodiments, the sugar-free formula contains no artificial flavors, no artificial colors, and no artificial sweeteners. Some embodiments may be gluten-free.

In one embodiment, an example formula contains a comprehensive profile of vitamins, probiotics, natural boosting minerals, anti-oxidants, super foods and natural purifiers along with aloe vera to boost bio-availability. Some examples of the ingredients include glutathione, beta-alanine, and alpha-lipoic acid.

In some embodiments, the formula is delivered as microencapsulated powder. A user adds water or other liquid to the powder and mixes by shaking or stirring before consuming.

What is claimed is:

1. A nutraceutical compound comprising, per serving, a blend of ingredients including:
cysteine;
glycine;
glutamic acid
beta alanine;
collagen peptides;
a probiotic blend; and
at least one B vitamin.

2. The nutraceutical compound of claim 1, further comprising alpha-lipoic acid.

3. The nutraceutical compound of claim 1, further comprising at least one type of superfood.

4. The nutraceutical compound of claim 1, further comprising zinc picolinate.

5. The nutraceutical compound of claim 1, further comprising selenomethionine.

6. The nutraceutical compound of claim 1, further comprising aloe vera leaf powder.

7. The nutraceutical compound of claim 1, further comprising watermelon fruit powder.

8. The nutraceutical compound of claim 1, further comprising blueberry fruit powder.

9. The nutraceutical compound of claim 1, wherein the nutraceutical compound is delivered as a microencapsulated powder.

10. The nutraceutical compound of claim 1, wherein the serving size is about 20 grams to 40 grams.

11. A nutraceutical compound comprising, per serving, a blend of ingredients including:
cysteine;
glycine;
glutamic acid;
beta alanine;
isoleucine;
leucine;
valine;
a probiotic blend;
malic acid;
bromeleain;
N-acetyl L-carnitine;
aloe vera leaf powder;
spirulina;
ginger root powder;
carrot powder;
fruit powder;
chlorella powder;
kelp powder;
4-aminobenzoic acid; and
quercetin.

12. The nutraceutical compound of claim 11, wherein the nutraceutical compound is delivered as a microencapsulated powder.

13. The nutraceutical compound of claim 11, wherein the serving size is about 20 grams to 40 grams.
